(19) United States
(12) Patent Application Publication
(10) Pub. No.: US 2011/0092788 A1
(43) Pub. Date: Apr. 21, 2011

(54) Systems And Methods For Providing Guidance In Administration Of A Medicine

(75) Inventors: James R. Long, Fishers, IN (US); Alan M. Greenburg, Indianapolis, IN (US); John F. Price, Mc Cordsville, IN (US); Eric S. Carlsgaard, Zionsville, IN (US); Paul J. Galley, Indianapolis, IN (US)

(73) Assignee: ROCHE DIAGNOSTICS OPERATIONS, INC., Indianapolis, IN (US)

(21) Appl. No.: 12/579,714

(22) Filed: Oct. 15, 2009

(51) Int. Cl. A61B 5/145; A61B 5/00; G09G 5/00

(52) U.S. Cl. 600/365; 600/300; 345/635

(57) ABSTRACT

A method of providing guidance using a portable hand-held electronic device in administration of a medicine by a patient is provided. The method includes acquiring image data of a first container of a first medicine prescribed to the patient and acquiring image data of a second container of a second medicine prescribed to the patient. The second container is different visually from the first container. The image data of the first container and the second container is stored in memory of the electronic device. On the portable hand-held electronic device, an instruction is processed to administer at least one of the first medicine and the second medicine to the patient. The stored image data is retrieved from memory corresponding to the at least one of the first medicine and the second medicine to be administered in the instruction. The retrieved image data is displayed on a display of the electronic device.

### TECHNICAL FIELD

The present invention relates generally to systems and methods for providing guidance in administration of a medicine, and in particular, systems and methods for providing such guidance using a portable electronic device.

### BACKGROUND

Insulin may usually be administered through subcutaneous injections, e.g., using syringes with needles, insulin pumps, insulin pens with needles, etc. Administration schedules may attempt to mimic the physiologic secretion of insulin by the pancreas. In these cases, both a long-acting insulin and a short-acting insulin may be used.

It is important to administer the proper insulin. Cases where an improper insulin was administered (e.g., short-acting insulin rather than long-acting insulin) are well-documented. For example, “I injected 46 Units of the Wrong Insulin” by Ann Gann at www.diabeteshealth.com details an instance where the author, living with type 2 diabetes for over thirteen years and a diabetes trainer, mistakenly injects herself with 46 units of short-acting Apidra instead of long-acting Lantus. In this article, the author recognizes, “The flaw of a routine activity, however, is that it is so very routine: you go through the motions without thinking.” The author picked the wrong insulin vial while at a hotel on a speaking trip. Irl B. Hirsch, MD in “Insulin Packaging: A Medical Error Waiting to Happen” available at docnews.diabetesjournals.org refers to an instance where an elderly patient accidentally administers 32 units of insulin aspart, a short-acting insulin analog, instead of the ordered insulin glargine, a basal or long-acting insulin. The author comments that such incidents are becoming more common and are likely often caused by similar packaging for different medications.

Low blood sugar is the most common adverse effect associated with insulins. Symptoms associated with low blood sugar may be different from patient-to-patient and may change over time. Severe low blood sugar may cause seizures and be life-threatening.

Aside from the physical adverse effects of improper insulin administration, there may also be emotional effects, such as stress and panic. In instances where a parent administers insulin to a child, an insulin dosing mistake can result in feelings of guilt and embarrassment. In a document “What to Do: Gave the Wrong Dose of Insulin” provided by Texas Children's Hospital at www.texaschildrenshospital.org, it is acknowledged that “We all make mistakes in managing diabetes at some time.” Accordingly, there is a long-felt need to help patients self-administer the proper insulin.

Various techniques have been employed to help patients differentiate between long-acting insulin and short-acting insulin. In the past, short-acting insulins were a clear liquid and long-acting insulins were cloudy. In 2001, however, insulin glargine was introduced, which is a long-acting insulin that is clear rather than cloudy. Thus, it is no longer reliable to distinguish between long-acting and short-acting insulins based on the appearance of the insulin itself. Some companies, such as Eli Lilly and Company have introduced color differentiation systems to help differentiate between different insulin types. It is also known to provide insulin vials of different shapes for different insulin types.

The efficacy of such approaches have been limited. Dr. Hirsch submits that even “most providers who prescribe insulin are not familiar with the differences in appearance of various insulin vials and labels.” Dr. Hirsch chronicles a test where he asked thirteen general internists, all with numerous patients having type 2 diabetes, to identify three types of insulin with the labels on the vials removed. Only three of the thirteen internists knew each insulin type, while five were aware that glargine comes in a tall, skinny vial.

Patients have varying ability to distinguish between vial labels and shapes. For example, it is recognized in “The Effects of a Diabetes Education Program on Clinical Outcomes in Patients with Diabetes” by Stefan Cadag et al. (Diabetes, JUN 2009, vol. 58 no. Suppl. 1, p. A241-A242, ISSN: 0012-1797) that an intervention through a diabetes education program can be highly effective in improving glycemic control. However, not all patients attend diabetes education programs. Additionally, patients have varying degrees of cognitive skills. For example, some elderly patients may have trouble seeing differences between labels and vial shapes. Thus, it is desirable to provide to provide additional guidance in the administration of insulin and to reduce confusion as to which vial of insulin to use.

### SUMMARY

In one embodiment, a method of providing guidance using a portable hand-held electronic device in administration of a medicine by a patient is provided. The method includes acquiring image data of a first container of a first medicine prescribed to the patient and acquiring image data of a second container of a second medicine prescribed to the patient. The second container is different visually from the first container. The image data of the first container and the second container is stored in memory of the electronic device. On the portable hand-held electronic device, an instruction is processed to administer at least one of the first medicine and the second medicine to the patient. The stored image data is retrieved from memory corresponding to the at least one of the first medicine and the second medicine to be administered in the instruction. The retrieved image data is displayed on a display of the electronic device.

In another embodiment, a method of providing guidance using a portable hand-held electronic device in administration of a medicine by a patient is provided. The method includes providing a connection between the portable hand-held electronic device including mobile phone capabilities and a blood glucose meter. The portable hand-held electronic device acquires blood glucose information from the blood glucose meter. On the portable hand-held electronic device, an instruction is processed to administer at least one of a short-acting insulin and a long-acting insulin to the patient based, at least in part, on the blood glucose information. Vial image data is retrieved from memory corresponding to the at least one of the short-acting insulin and the long-acting insulin to be administered in the instruction. The retrieved vial image data is displayed on a display of the electronic device.

In another embodiment, a data and communications system for providing guidance in administration of a medicine by a patient includes a mobile device comprising a memory having a first vial image and a second vial image stored in the memory. The first vial image corresponds to a vial of short-acting insulin for administration by the patient and the second vial image corresponds to a vial of long-acting insulin for administration by the patient. The mobile device further includes a processor including logic that processes on the portable hand-held electronic device an instruction to administer at least one of the short-acting insulin and the long-acting insulin to the patient. At least one of the first vial image and the second vial image is retrieved from memory corresponding to the at least one of the short-acting insulin and the long-acting insulin to be administered in the instruction. The at least one of the first vial image and the second vial image corresponding to the at least one of the short-acting insulin and the long-acting insulin to be administered in the instruction is displayed on a display of the electronic device.

These and other advantages and features of the invention disclosed herein, will be made more apparent from the description, drawings and claims that follow.

### BRIEF DESCRIPTION OF THE DRAWINGS

The following detailed description of the embodiments of the present invention can be best understood when read in conjunction with the following drawings, where like structure is indicated with like reference numerals, and in which:

FIG. 1 is a system view of an embodiment of a data and communications system for providing guidance in self-administration of a medicine;

FIG. 2 is a schematic view of an embodiment of a mobile device for use in the data and communications system of FIG. 1;

FIG. 3 is a schematic view of an embodiment of a blood glucose meter for use in the data and communications system of FIG. 1;

FIGS. 4 and 5 illustrate embodiments of systems and methods for acquiring image data of a medicine container;

FIGS. 6 and 7 illustrate other embodiments of systems and methods for acquiring image data of a medicine container;

FIGS. 8 and 9 illustrate an embodiment of information collection and providing an instruction to a user including image data;

FIG. 10 illustrates an embodiment of a display including image data and patient instruction; and

FIG. 11 illustrates another embodiment of a display including image data and patient instruction.

### DETAILED DESCRIPTION

The following description of the preferred embodiment is merely exemplary in nature and is in no way intended to limit the invention or its application or uses.

Embodiments described herein generally relate to systems and methods that provide guidance in administering medicine to a patient. As used herein, the term “patient” refers to a person who is under medical care or treatment. In particular embodiments, the systems and methods are used to verify that the correct medicine is picked for administration to the patient using, in part, visual differences between different medicine containers. As one example, which will be described in greater detail below, a diabetic patient may typically self-administer his or her own insulin. Depending on a number of conditions, the patient may administer a long-acting insulin or a short-acting insulin. The systems and methods described herein may provide guidance that the proper insulin vial is chosen for administration of the proper insulin.

A flexible insulin therapy may include administration of long-acting insulin and short-acting insulin. As used herein, the terms “long-acting” and “basal” may be used interchangeably and the terms “short-acting,” “rapid-acting” and “bolus” may be used interchangeably. Long-acting insulin may be a background insulin and is typically administered when the patient is not eating. Long-acting insulin is usually present in the blood stream of the patient for long periods of time to assist in movement of glucose into cells. Short-acting insulin may be administered to bring down high blood sugar. Short-acting insulin may be administered, for example, to cover carbohydrates ingested during a meal.

In some embodiments, insulin administration may be adjusted based on, among other things, current blood glucose levels. The process of monitoring one's own blood glucose with a blood glucose meter is often referred to as self-monitoring of blood glucose (SMBG). In some cases, a portable hand-held blood glucose meter, among other devices, may be used in SMBG, which may be a relatively small, battery-operated device.

To test glucose with a blood glucose meter, a small sample of blood may be placed on a disposable test strip. The portable hand-held blood glucose meter may include a strip port that receives the disposable test strip. The test strip may be coated with chemicals (glucose oxidase, dehydrogenase, or hexokinase) that combine with glucose in blood. The portable hand-held blood glucose meter then measures concentration of glucose in the blood sample. The portable hand-held blood glucose meter then displays the glucose concentration as a number (or glucose measurement value). This glucose measurement value may be used in selecting whether to administer a long-acting insulin, a short-acting insulin or both and the dosage.

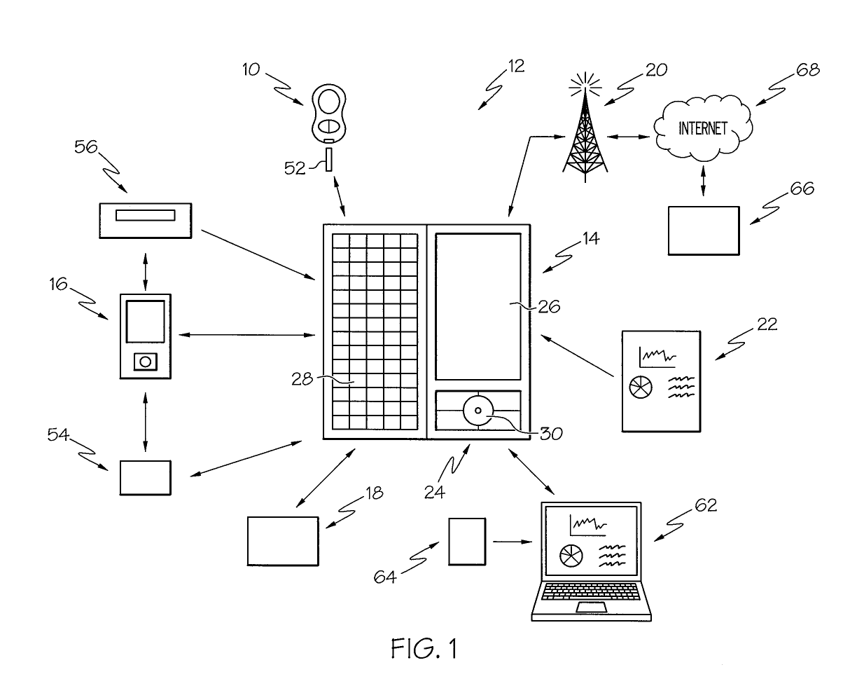

Referring to FIG. 1, in some embodiments, the blood glucose meter 10 may be part of a data and communications system 12 that provides a patient guidance for self-monitoring of diabetes and health management. The system 12 may include a mobile device 14 that interacts (e.g., wired and/or wirelessly) with one or more external systems, such as the blood glucose meter 10, a hand-held/portable computer 16, a health monitor 18, among others. Communication between the various devices may be achieved by any suitable connection, such as using Bluetooth, infrared, wired connections (e.g., USB), etc. In the illustrated embodiment, the mobile device 14 may include mobile phone capabilities for mobile voice or data communication over a network of a phone provider 20. Through the mobile phone capabilities (meeting standards for wireless communication such as third generation (3G), fourth generation (4G), etc.), the mobile device 14 may be capable of, for example, voice functions, telephone functions and additional services and accessories, such as SMS for text messaging, e-mail, packet switching for access to the Internet, gaming, Bluetooth, infrared, camera with video recorder and MMS for sending and receiving photos and video, MP3 player, radio and GPS. The mobile device 14 may also include computing capabilities for running software 22 for data analysis. The mobile device 14 may include a user interface 24, which may include a touch screen and/or display 26, a touch pad or keyboard 28, which may provide a QWERTY layout in one embodiment, a AZERTY or QWERTZ layout in other embodiments, and navigational buttons 30.

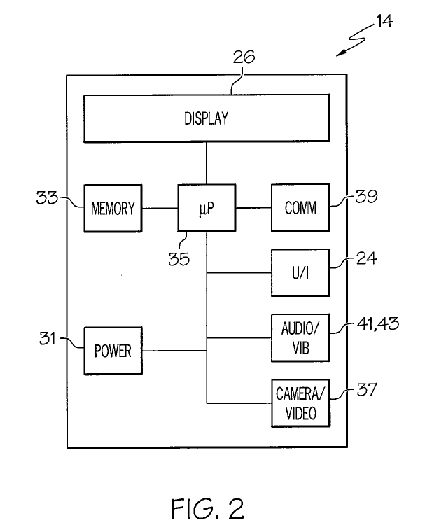

Referring briefly to FIG. 2, the mobile device 14 may generally include a power supply 31, the display 26, memory 33, a microprocessor 35, the user interface 24 and a camera/video module 37 providing camera and video capturing capabilities. The mobile device 14 may also include audio 41 and vibration devices 43 and a communications module 39 providing the telephone services and communication to the other devices, such as the blood glucose meter 10.

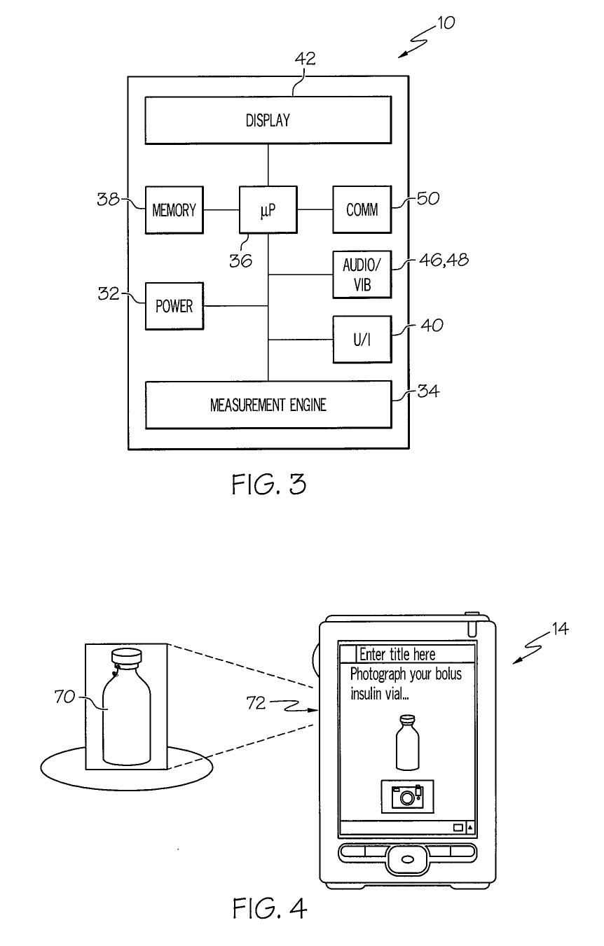

Referring back to FIG. 1, in addition to mobile phone capabilities, the mobile device 14 may interact with a number of external systems and devices, such as the blood glucose meter 10. The blood glucose meter 10 may be a self-monitoring blood glucose meter. An example of a blood glucose meter is an Accu-Chek® Aviva described in the booklet “AccuChek® Aviva Blood glucose meter Owner's Booklet” (2007), portions of which are disclosed in U.S. Pat. No. 6,645,368 assigned to Roche Diagnostics Operations, Inc., the details of which are hereby incorporated by reference in their entirety. Referring briefly to FIG. 3, the blood glucose meter 10 may include a power supply 32, measurement engine 34, microprocessor 36, memory 38, user interface 40 and display 42. Some embodiments of the blood glucose meter 10 may include features such as diabetes software run by processor 36, an audio device 46, a vibrator device 48 and communications module 50, e.g., for communication with the mobile device 14.

Referring again to FIG. 1, in some embodiments, the blood glucose meter 10 may be used in connection with the mobile device 14 to perform actions such as prompt the patient to take an action, acquire a data event and/or perform calculations on information. An example of a blood glucose meter combined with a hand held computer is disclosed in U.S. patent application Ser. No. 11/424,757 filed Jun. 16, 2006 entitled “System and Method for Collecting Patient Information from which Diabetes Therapy may be Determined,” the details of which are hereby incorporated by reference in their entirety. Test strips 52, also known as disposable biosensors, may be used with the blood glucose meter 10 to receive a sample of blood, which is exposed to an enzymatic reaction and may be measured by electrochemistry techniques, optical techniques, or both to measure blood glucose. An exemplary test strip and measurement engine are disclosed in U.S. patent application Ser. No. 10/871,673 entitled “Reagent Stripe for Test Strip” filed Jan. 27, 2005, the details of which are incorporated by reference as if fully set forth herein.

A continuous glucose monitor 54 may be used to obtain time-resolved data and communicate the data to the mobile device 14 to identify fluctuations and trends that may otherwise go unnoticed with spot monitoring of blood glucose levels and standard HbA1c tests, such as low overnight glucose levels, high blood glucose levels between meals, early morning spikes in blood glucose levels, and how diet and physical activity affect blood glucose along with the effect of therapy changes. An example of a continuous glucose monitor 54 is described in U.S. Pat. No. 7,389,133, which is hereby incorporated by reference.

In some embodiments, the patient may have an implanted or externally worn infusion device 56 that is configured to deliver a glucose-lowering drug, e.g., insulin, to the patient. In such cases, the liquid infusion device 56 may include a communication circuit that may be configured for connection with mobile device 14, the blood glucose meter 10 and/or the portable computer 16. In embodiments wherein the infusion device 56 is an externally-worn liquid infusion device, the communication circuit may be configured for hard wire communications and/or wireless communications with any of the devices 10, 14 and 16. In other embodiments wherein the liquid infusion device 56 is an implanted infusion device, communications between the device 56 and any of the devices 10, 14 and 16 may generally be carried out via the wireless communication link. Liquid infusion information, e.g., insulin delivery information, may be automatically transferred from the liquid infusion device 56 to the blood glucose meter 10, to a processor of the mobile device 14 and/or to a processor portable computer 16. As used herein, the term “insulin delivery information” includes any information relating to delivery of insulin to the patient including, for example, but not limited to, insulin delivery type, e.g., basal, correction bolus or meal compensation bolus, insulin quantity or amount, insulin delivery pattern, e.g., single or multiple delivery events, and insulin delivery rates (e.g., speed of delivery of the one or more insulin delivery events). In embodiments that do not include a liquid infusion device 56, an insulin or another blood glucose lowering drug may be instead delivered to the patient via manual injection or other manual administering technique. The insulin delivery information may alternatively be manually provided to the mobile device 14 via the user interface 24, and may alternatively or additionally be manually provided to the portable computer 16.

In some embodiments, additional monitoring devices 18 may be used to obtain health-related data. Such monitoring devices 18 may include devices for determining blood pressure, weight, etc. The monitoring devices 18 may communicate such data to the mobile device 14.

In some embodiments, the mobile device 14 may communicate with a personal computer 62, for example, running diabetes management software 64. The diabetes management software 64 may be used to collect self-care data and analyze results using graphs and charts for continuous improvement of diabetes management. Customized reports may be generated related to blood glucose, ketones, blood pressure, cholesterol, weight, carbohydrates and exercise, as examples.

The mobile device 14 may also be configured to utilize a diabetes management and collection system 66 via the Internet 68 using services provided by phone provider 20. For example, web-based applications may be accessible over the Internet. Charts, reports and other data may be communicated over the Internet 68 to and from the mobile device 14 for assistance in diabetes management. In some embodiments, clinicians can use diabetes software to evaluate patient diabetes data for therapy results, for example, sent over the Internet using the mobile device 14. An example of diabetes software is disclosed in U.S. patent application Ser. No. 11/999,968 filed Dec. 7, 2007 entitled “Method and System for Setting Time Block,” the details of which are hereby incorporated by reference in their entirety.

The data and communications system 12 may provide insulin delivery information to the patient and provide guidance that the proper insulin vial is chosen for administration. The data and communications system 12 may be used to determine any of insulin delivery type, e.g., basal, correction bolus or meal compensation bolus, insulin quantity or amount (e.g., in international units or I.U.), insulin delivery pattern, e.g., single or multiple delivery events, and insulin delivery rates (e.g., speed of delivery of the one or more insulin delivery events). In instances where a specific type of insulin is specified (e.g., between short-acting and long-acting insulin), the data and communications system 12 may provide an output to aid the patient in selecting the proper vial from which to draw the insulin for self-administration.

As one illustrative example of data collection, the mobile device 14 may have memory 33 having at least one algorithm stored therein that is executable by the processor 35 of the mobile device to present instructions to the patient via the display 26, a speaker configured to present audible instructions to the patient, etc (see FIG. 2). The mobile device 14 in this implementation may include the keypad or keyboard 28, a touch screen, etc. in which case the patient may manually enter at least a portion of the patient information. Patient glucose information may be manually entered into the mobile device 14 or may alternatively be transferred automatically from the blood glucose meter 10 to the mobile device. Weight, blood pressure and other patient information may be communicated to the mobile device 14 via the monitoring devices 18. Insulin delivery information may likewise be manually or automatically transferred to the mobile device 14 via the liquid infusion device 56 in embodiments that include the liquid infusion device.

The mobile device 14 may be programmed with one or more algorithms that are executable by the processor 35 of the mobile device to guide a patient through a patient information collection time period. Initially, the patient information entered into the mobile device 14 may be stored in the memory 33 of the mobile device. The patient information may be subsequently transferred or copied to the diabetes management and collection system 66 for storage in a database, e.g., via the Internet 68 and/or the telephone network 20. In some embodiments, a health care professional may access the patient information stored in the database of the diabetes management and collection system 66 to analyze this data from a remote location and design a diabetes therapy for the patient, or modify an existing diabetes therapy, that is based on this analysis.

While the mobile device 14 may be programmed to guide a patient through a patient information collection time period, other devices of the data and communications system 12 may be programmed to guide the patient through the patient information collection. For example, any one or more of the portable computer 16, personal computer 62, blood glucose meter 10 and diabetes management and collection system 66 may be by used to guide the patient through the patient information collection and/or store the patient information in memory. Various methods of patient information collection is disclosed in U.S. patent application Ser. No. 11/424,757, already incorporated by reference.

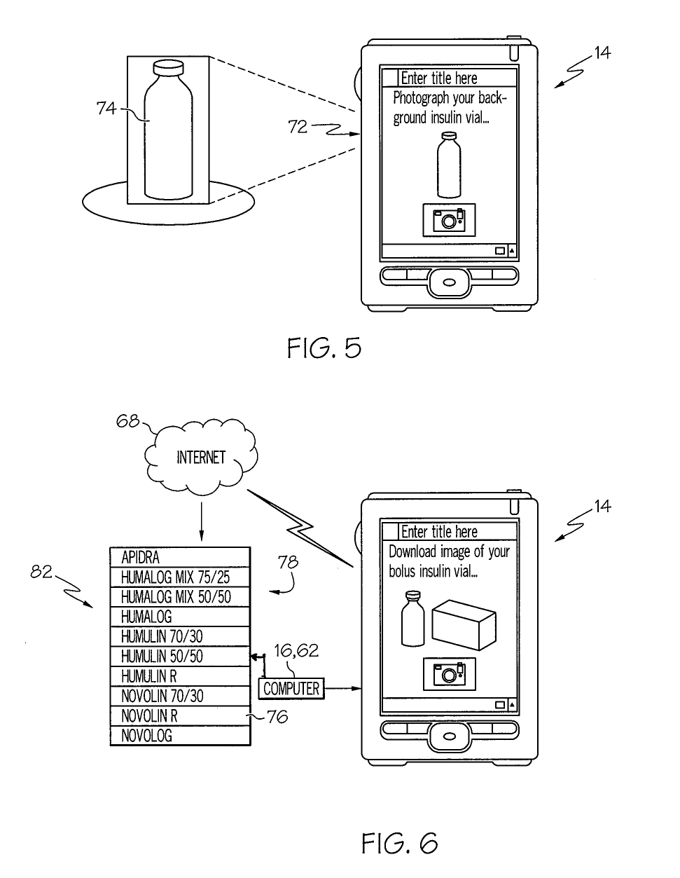

As part (or separate from) the patient information collection, referring now to FIG. 4, image data may be acquired corresponding to a vial 70 (e.g., or other container) of insulin. The vial 70 may contain an insulin prescribed to the patient. As indicated above, in some instances, a patient may be prescribed more than one insulin type, each type having its own vial. In these instances, image data may be acquired corresponding to multiple insulin vials (e.g., short-acting and long-acting). In many embodiments, the different vials will be visually different from each other to aid the patient in selecting the proper vial. FIG. 4 illustrates steps of acquiring image data of a bolus insulin vial 70 using a camera 72, for example, built into the mobile device 14. The image data may be acquired through patient operation of the camera 72 of the mobile device 14. The image data may be saved in memory 33 of the mobile device 14 and/or transferred or copied to one or more of the other devices. The bolus insulin vial image data may also be associated with any instructions involving the bolus insulin. FIG. 5 illustrates steps of acquiring image data of a basal or background insulin vial 74 using the camera 72, which may be saved in memory 33 of the mobile device 14 and/or transferred or copied to one or more of the other devices. The background insulin vial image data may also be associated with any instructions involving the background insulin. Other devices of the data and communications system 12 (e.g., the blood glucose meter 10, portable computer 16, personal computer 60, etc.) may include a camera for use in acquiring image data. As can be seen by comparing FIGS. 4 and 5, the bolus insulin vial 70 looks different than the background insulin vial 74.

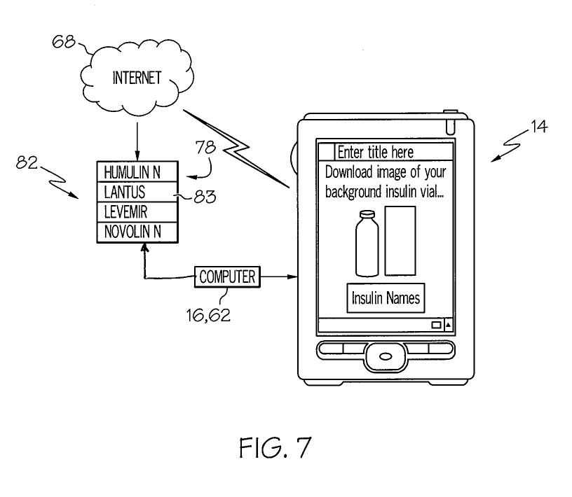

Referring to FIGS. 6 and 7, image data of the insulin vials 70 and 74 may be acquired from remote memory using device 14. Referring first to FIG. 6, as part of the patient information collection, an image data file 76 may be selected from a database 78 of image data files. The image data files may be organized in separate lists, for example, a first list containing only short-acting insulin products and a second list containing only long-acting insulin products or the lists may be combined into a single list containing both long and short-acting insulin products. In one embodiment, the database 78 may be part of the diabetes management and collection system 66 and be accessible over the Internet 68. When instructed by the patient via the user interface 24, such as when initially launching a software application running on device 14, which facilitates therapy guidance such as, for example, method 90 that is discussed in later sections, and/or according to a schedule configured by the one or more algorithms that are executable by the processor 35 of the mobile device to guide the patient through the patient information collection time period, the processor of the device 14 communicates with the diabetes management and collection system 66 and requests list 82 containing a current listing of the image files for the insulin vials contained in the data base 78. Upon receiving the request from the processor, the diabetes management and collection system 66 sends the current list 82 to the device 14 over the Internet 68, which in one embodiment after being received by the device 14 is stored in memory 33 for later accessing by the processor 35 according to the schedule. For example, the schedule may be dictated by processor 35 configured by the one or more algorithms to help the patient manage a prescribed therapy, e.g., which causes the processor of the mobile device 14 to prompt the patient on the display to download an image of a particular type of insulin if such had not previously been done. Thus, when indicated by the schedule or after receiving the list 82 from the diabetes management and collection system 66 after a request is made by the patient, the list 82 is displayed on display 26 by the processor 35. The patient then uses the user interface 24 to select the image data file 76 associated with the proper bolus insulin vial 70 from the displayed list 82 in FIG. 6, whereby the processor 35 sends the selection made by the user to the diabetes management and collection system 66. The selected image data file 76 is then sent from the database 78 by the diabetes management and collection system 66 over the Internet 68 to the mobile device 14, after which the processor 35 then displays on the display 26 for confirmation by the patient. If the displayed image data file 76 for the bolus insulin vial 70 is correct, after confirmation by the patient indicating acceptance via the user interface 24, the image data file 76 is stored in memory 33 for later retrieval by the processor 35 as will be explained hereafter with regard to method 90. The above selection process is repeated for all remaining insulin vials. For example, FIG. 7 illustrates selection of the image data file 83 associated with the proper background insulin vial 74 from list 82, for example, when prompted as explained above. In the embodiments of FIGS. 6 and 7, the image data may be created by someone other than the patient. In these embodiments, guidelines may be developed for generating the image data. For example, a preselected zoom factor may be used. The image data may include both the insulin vial and the corresponding package, as shown in FIGS. 6 and 7. In some embodiments, as represented by FIGS. 6 and 7, the computer 16 and/or 62 may include the image data file 76 and database 78 of image data files.

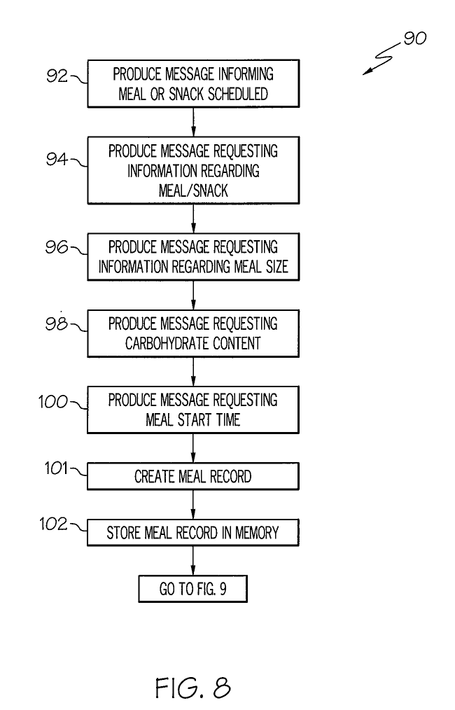

Referring to FIG. 8, an exemplary instruction method 90 is shown to illustrate use of the image data. In a preferred embodiment, the method 90 is implemented as a software application having program instructions which cause the processor 35 of the mobile device 14 to implement the following technical features. In this exemplary embodiment, at step 92, the processor 35 of the mobile device 14 produces a message via the display 26 informing the patient that a meal or snack is scheduled where the scheduled time corresponds to the time at which the patient is scheduled to consume the meal or snack. At step 94, the processor 35 of the mobile device 14 produces a message instructing the patient to record information relating to the pending meal or snack. The processor 35 of the mobile device 14 may instruct the patient to enter a meal type MT, via the user interface 24. The patient may enter a meal type or be presented on the display 26 with a menu, which the processor 35 has accessed from memory 33, from which to choose the meal type. The patient's choices may include breakfast B, lunch L, dinner D, and snack S. At step 96, the processor 35 of the mobile device 14 may instruct the patient via the display 26 to enter a meal size MS, via the user interface 24. The patient may enter a meal size or be presented with a menu, which the processor 35 of the mobile device 14 has accessed from memory 33, from which to choose a meal size. The patient's choices for meal size may include, for example, small S, medium M, and large L. At step 98, the processor 35 of the mobile device 14 instructs the patient via the display 26 to enter an estimated carbohydrate content CC, of the meal via the user interface 24. Thereafter at step 100, the processor 35 of the mobile device 14 instructs the patient via the display 26 to enter the time TM, that the meal begins via the user interface 24. The mobile device 14 in step 101 creates a meal record. Illustratively, the meal record may take the form of [MT, MS, CC, TM, DATE, TIME] where MT is the meal type, e.g., B, L, D or S, MS is the meal size, e.g., S, M, L, CC is the estimated carbohydrate content of the meal, TM is the time the meal began or will begin, DATE is the current calendar and TIME is the current time of day. At step 102, the processor 35 of the mobile device 14 stores the meal record in memory 33.

It should be understood that the method 90 may be modified to require the patient to enter more or less meal-related information, and examples of additional meal information that may be required to be entered by the patient include, but are not limited to, a meal speed value, corresponding to the speed at which the meal is consumed, a total glycemic index of the meal, and meal size in terms of fat content, carbohydrate content and protein content. The term “glycemic index” is defined for purposes of this document as a parameter that ranks meals and snacks by the speed at which the meals or snacks cause the patient's blood sugar to rise. Thus, for example, a meal or snack having a low glycemic index produces a gradual rise in blood sugar whereas a meal or snack having a high glycemic index produces a fast rise in blood sugar. One exemplary measure of total glycemic index may be, but should not be limited to, the ratio of carbohydrates absorbed from the meal and a reference value, e.g., derived from pure sugar or white bread, over a specified time period, e.g., 2 hours. With any of the meal size or meal speed information, it will be understood that method 90 may be configured to require a patient to enter absolute estimates as illustrated e.g., “small,” or may alternatively be configured to require the patient to enter such information in relative terms, e.g., “smaller than normal.”

Referring to FIG. 9, following completion of method 90, the program instructions cause the processor 35 of the mobile device 14 at step 104 to produce a message via the patient interface instructing the patient to take and record a patient glucose measurement prior to consuming the scheduled meal or snack. Following step 104, the processor 35 of the mobile device 14 computes at step 106 a bolus amount based, at least in part, on the meal information just entered by the patient, the patient glucose measurement and on a predefined glucose target. Thereafter at step 108, the processor 35 of the mobile device 14 produces a message via the patient interface instructing the patient to inject, or otherwise deliver, insulin in the amount of the bolus amount computed at step 104. The processor 35 of the mobile device 14 also displays at step 110 the proper bolus insulin vial 70 associated with the bolus insulin instruction, for example, along with the instructions.

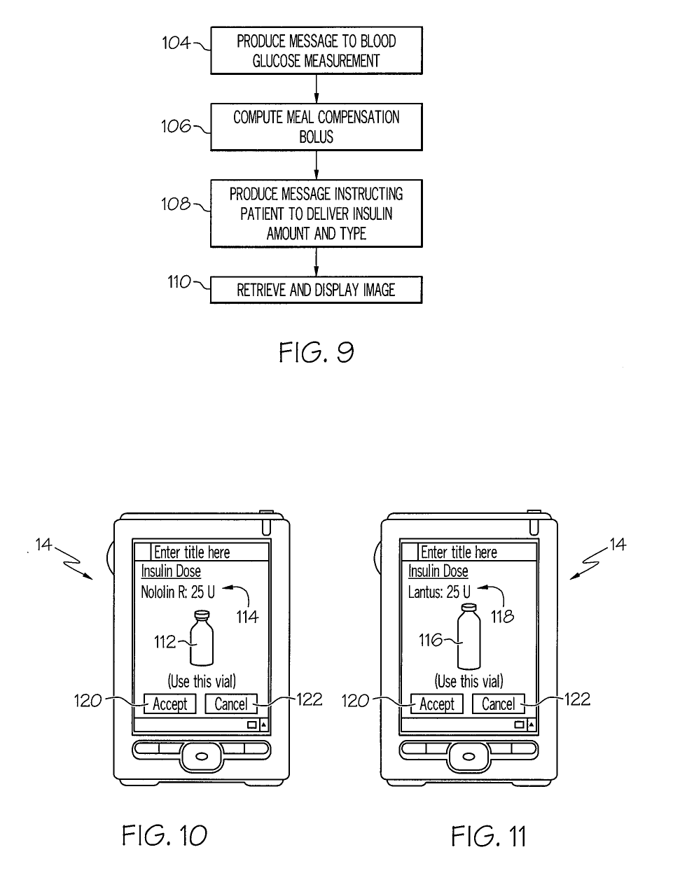

As one example, referring to FIGS. 10 and 11, the processor 35 of the mobile device 14 is illustrated displaying a short-acting insulin image 112 with insulin dosing information 114 (FIG. 10) and a long-acting insulin image 116 with insulin dosing information 118 (FIG. 11). The insulin images 112 and 116 are displayed on the display 26. In some embodiments, an interactive element (e.g., buttons 120 and 122) may be displayed on the display 26, for example, prompting the patient to interact with the mobile device 14.

While FIGS. 10 and 11 illustrate the vial images being displayed on the mobile device 14, they may be displayed on other suitable devices, such as the portable computer 16, personal computer 62 and the blood glucose meter 10, for example, depending on which of the devices the patient is relying on for delivering the administration instructions. Additionally, the computer 16 and/or 62 may be used to store and provide patient information and/or schedule information and provide this information to the patient and/or the mobile device 14. Displaying of the proper insulin vial associated with a particular instruction can reduce the probability of incorrect insulin by reminding the patient of what the insulin vial looks like associated with the particular dosing instruction. The image data can be part of the dosing instruction and/or be displayed in a wallpaper fashion. In some embodiments, the image data may be displayed on one or more of the devices of the data and communications system, e.g., simultaneously. While insulin vials are described above, other insulin delivery devices may be displayed such as insulin pens, particularly, where different insulin pens are used by the patient that are visually different. Color schemes and/or icons associated with a particular medication may be reinforced, for example, highlighted in the images so an association can be made between a particular medication and its representation, for example, in diary lists, reports and graphs.

The lists of image data files and associate images may be regional and/or country specific. The lists of image data files may be updated to include new insulin products. In some embodiments, multiple images may be associated with a particular insulin product, for example, cartons, vials, insulin pens, cartridges, etc.

All documents cited herein are incorporated herein by reference and the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

The above description and drawings are only to be considered illustrative of exemplary embodiments, which achieve the features and advantages of the present invention. Modification and substitutions to specific process steps, system, and setup can be made without departing from the spirit and scope of the present invention. Accordingly, the invention is not to be considered as being limited by the foregoing description and drawings, but is only limited by the scope of the appended claims.

What is claimed is:

1. A method of providing guidance using a portable hand-held electronic device in administration of a medicine by a patient, the method comprising:
acquiring image data of a first container of a first medicine prescribed to the patient;
acquiring image data of a second container of a second medicine prescribed to the patient, the second container being different visually from the first container;
storing the image data of the first container and the second container in memory of the electronic device;
processing on the portable hand-held electronic device an instruction to administer at least one of the first medicine and the second medicine to the patient;
retrieving from memory the stored image data corresponding to the at least one of the first medicine and the second medicine to be administered in the instruction; and
displaying the retrieved image data on a display of the electronic device.

2. The method of claim 1, wherein the image data of at least one of the first and second container is generated using a camera.

3. The method of claim 2, wherein the portable hand-held electronic device comprises the camera.

4. The method of claim 1, wherein the image data of at least one of the first and second container is acquired from a database over the Internet.

5. The method of claim 1, wherein the image data of at least one of the first and second container is acquired from a computer database.

6. The method of claim 1, wherein the step of displaying the retrieved image data includes displaying the instruction simultaneously with the retrieved image data on the display of the electronic device.

7. The method of claim 1 further comprising entering patient information into the portable hand-held electronic device.

8. The method of claim 7, wherein the patient information includes blood glucose information received from a blood glucose meter.

9. A method of providing guidance using a portable hand-held electronic device in administration of a medicine by a patient, the method comprising:
providing a connection between the portable hand-held electronic device including mobile phone capabilities and a blood glucose meter, the portable hand-held electronic device acquiring blood glucose information from the blood glucose meter;
processing on the portable hand-held electronic device an instruction to administer at least one of a short-acting insulin and a long-acting insulin to the patient based, at least in part, on the blood glucose information;
retrieving container image data from memory corresponding to the at least one of the short-acting insulin and the long-acting insulin to be administered in the instruction; and
displaying the retrieved container image data on a display of the electronic device.

10. The method of claim 9, wherein the container image data corresponding to the at least one of the short-acting insulin and the long-acting insulin is generated using a camera.

11. The method of claim 10, wherein the portable hand-held electronic device comprises the camera.

12. The method of claim 9, wherein the container image data corresponding to the at least one of the short-acting insulin and the long-acting insulin is acquired from a database over the Internet.

13. The method of claim 9, wherein the step of displaying the retrieved container image data includes displaying the instruction simultaneously with the retrieved container image data on the display of the electronic device.

14. A data and communications system for providing guidance in administration of a medicine by a patient, the system comprising:
a mobile device comprising a memory having a first container image and a second container image stored in the memory, the first container image corresponding to a short-acting insulin for administration by the patient and the second container image corresponding to a long-acting insulin for administration by the patient, the mobile device further including a processor including logic that
processes on the portable hand-held electronic device an instruction to administer at least one of the short-acting insulin and the long-acting insulin to the patient;
retrieves from memory at least one of the first container image and the second container image corresponding to the at least one of the short-acting insulin and the long-acting insulin to be administered in the instruction; and
displays the at least one of the first container image and the second container image corresponding to the at least one of the short-acting insulin and the long-acting insulin to be administered in the instruction on a display of the electronic device.

15. The system of claim 14, wherein the mobile device comprises a mobile phone and a camera that is used to generate the at least one of the first container image and the second container image.

16. The system of claim 15, wherein the at least one of the first container image and the second container image is acquired from a database over the Internet.

17. The system of claim 14, wherein the processor displays the instruction simultaneously with the at least one of the first container image and the second container image on the display of the electronic device.

18. The system of claim 14 further comprising a blood glucose meter that sends blood glucose information to the mobile device.

19. The system of claim 14, wherein the mobile device comprises a blood glucose meter.

20. The system of claim 14, wherein the first container image is a first vial image and the second container image is a second vial image.
